(19) United States
(12) Patent Application Publication
(10) Pub. No.: US 2017/0172995 A1
(43) Pub. Date: Jun. 22, 2017

(54) Pharmaceutical compositions of Nicotine and process for preparation thereof

(71) Applicants: Venkateswara Rao Repaka, Hyderabad (IN); Venkata Ramana Goalla, Hyderabad (IN); Shashikanth Gujjaru, Hyderabad (IN); Sivakumaran Meenakshisunderam, Hyderabad (IN)

(72) Inventors: Venkateswara Rao Repaka, Hyderabad (IN); Venkata Ramana Goalla, Hyderabad (IN); Shashikanth Gujjaru, Hyderabad (IN); Sivakumaran Meenakshisunderam, Hyderabad (IN)

(21) Appl. No.: 15/265,765

(22) Filed: Sep. 14, 2016

(30) Foreign Application Priority Data
Sep. 8, 2015 (IN) IN 4746/CHE/2015

(51) Int. Cl. A61K 31/465; A61K 9/16; A61K 9/00

(52) U.S. Cl. CPC A61K 31/465; A61K 9/0056; A61K 9/1611

(57) ABSTRACT

The present invention relates to nicotine or nicotine polacrilex lozenge compositions comprising nicotine or nicotine derivatives as an active agent and the process for preparation thereof. The present invention also relates to method of using nicotine or nicotine polacrilex lozenge compositions for reducing withdrawal symptoms or tobacco usage particularly nicotine craving, associated with quitting smoking.

### CROSS REFERENCE TO RELATED APPLICATIONS

This application claims priority from an Indian Patent Application IN 4746/CHE/2015 filed Sep. 8, 2015.

### FIELD OF THE INVENTION

The present invention relates to pharmaceutical composition comprising nicotine or nicotine derivatives as an active agent, process of preparation thereof and method of using the same. Particularly, the present invention relates to pharmaceutical composition comprising nicotine or nicotine polacrilex, process of preparation thereof and method of reducing withdrawal symptoms or tobacco usage particularly nicotine craving, associated with quitting smoking.

### BACKGROUND OF THE INVENTION

Use of nicotine sustains tobacco addiction, which in turn causes devastating health problems including heart disease, lung disease, and cancer. Smoking harms almost every organ of the body. Quitting smoking at any age leads to significant reductions in the risks associated with smoking, and the vast majority of smokers indicate an interest in quitting. Despite these facts, however, approximately 80% of smokers who attempt to quit on their own relapse within the first month of abstinence and only about 3% remain abstinent at 6 months. This illustrates the powerful force of tobacco addiction and the chronic nature of the disorder. Tobacco addiction is best considered a chronic disease, with most smokers requiring repeated interventions over time before achieving permanent abstinence.

Cigarette smoke contains an extremely high concentration of oxidants. The reactive oxidant substances generated by smoking induce inflammation in the lung and its airway. Cigarette smoking causes an inflammatory process in the central airways, peripheral airways, and lung parenchyma, which is present even in smokers with normal lung function. Studies have shown that in bronchial biopsies obtained from central airways, smokers have chronic inflammatory changes, with increased numbers of specific inflammatory cell types in different parts of the lung, and structural remodeling resulting from repeated injury and repair. The exact role of smoking cessation in the airway inflammation process is still unknown. Cross-sectional studies suggest that there is still ongoing inflammation in the lung even after smoking cessation. The persistent inflammation in COPD may be, at least in part, related to repair of the smoke-induced tissue damage in the airways. It remains to be determined which parts of the observed inflammatory changes are beneficial and which are harmful.

Smoking cessation is challenging and behavioral interventions alone have had only modest success. As a result, pharmacotherapy has been increasingly relied on to assist in smoking cessation. The most common of these pharmacologic interventions has been nicotine replacement therapy (NRT).

Various forms of NRT have been used for smoking cessation in the form of a transdermal patch, gum, nasal spray, oral inhaler, and lozenges. These products supply low doses of nicotine and do not contain the toxins found in smoke. The goal of therapy is to cut down on cravings for nicotine and ease the symptoms of nicotine withdrawal.

All forms significantly improve quit rates. However lozenges are more helpful as they are easy to use, have minimal adverse effects (heartburn, hiccups, and nausea), provide faster dissolution time and onset of craving relief as nicotine is absorbed through the mouth into the bloodstream.

Nicotine is a natural alkaloid obtained from the dried leaves and stems of the Nicotiana tabacum and Nicotiana rustica, where it occurs in concentrations of 0.5-8%. It is a hygroscopic, oily, colorless or pale yellow liquid, which is miscible with water in its base form.

The chemical name of nicotine is (S)-3-(1-Methylpyrrolidin-2-yl) pyridine. Its molecular formula is C₁₀H₁₄N₂ and its molecular weight is 162.26 g/mol. The chemical structure is:

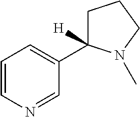

Nicotine polacrilex is a resin complex of nicotine and polacrilin, which is a cation-exchange resin prepared from methacrylic acid and divinylbenzene.

Nicotine is currently marketed as Commit®/Nicorette® 2 and 4 mg lozenges and mini lozenges used for reducing withdrawal symptoms, including nicotine craving, associated with quitting smoking. Nicorette lozenges are available in original, mint and cherry flavor. The original flavor lozenges contain the following ingredients: aspartame, calcium polycarbophil, flavor, magnesium stearate, mannitol, potassium bicarbonate, sodium alginate, sodium carbonate and xanthan gum.

U.S. Pat. No. 8,501,164 discloses oral lozenge composition comprising: a) a master granule component comprising: at least one an alkaline buffering agent; and at least one diluent; and b) an extragranular component blended with the master granule component comprising a nicotine active and at least one alkaline buffering agent.

U.S. Pat. No. 8,9407,72 discloses oral lozenge composition comprising: a) a master granule component comprising: at least one an alkaline buffering agent selected from the group consisting of sodium carbonate, sodium bicarbonate, potassium phosphate, potassium carbonate and potassium bicarbonate, and mixtures thereof; at least one dissolution modifier; and at least one diluent wherein the master granule component is obtained through wet or dry granulation; and b) an extragranular component blended with the master granule component comprising nicotine polacrilex and at least one alkaline buffering agent selected from the group consisting of sodium carbonate, sodium bicarbonate, potassium phosphate, potassium carbonate and potassium bicarbonate, and mixtures thereof.

US 2013/0177646 discloses solid pharmaceutical dosage form for the release of nicotine in the oral cavity comprising a core encapsulated by at least one film coating, wherein the core comprises nicotine and wherein the film coating comprises at least one film-forming polymer.

US 2011/0165253 discloses method for producing a nicotine-containing granulate comprising (a) mixing nicotine or a pharmaceutically acceptable nicotine derivative with an excipient; (b) compacting the mixture obtained in step (a) in a roller compactor to produce granules; and (c) screening the granules obtained in step (b) to remove particles.

US 2012/0244104 discloses solid oral transmucosal dosage forms containing a therapeutically effective amount of nicotine polacrilex, citric acid monohydrate, sucrose and liquid glucose.

US 20150105431 discloses oral lozenge composition comprising: a) a master granule component comprising: at least one an alkaline buffering agent; at least one dissolution modifier; and at least one filler; b) a nicotine active; and c) at least one alkaline buffering agent.

US 20160113868 discloses nicotine lozenge for oral administration comprising: an intragranular component comprising a water soluble, synthetic or semi-synthetic, non-ionic polymer; and an extragranular component comprising a nicotine active and at least one high viscosity water soluble, synthetic or semi-synthetic, non-ionic polymer and the at least one low viscosity water soluble, non-ionic, synthetic or semi-synthetic polymer.

The disclosures made in the prior art compositions comprise nicotine in the extragranular portion, and alkaline agent(s) either in the intragranular portion or both within the intragranular portion extragranular portion in order to achieve optimal oral pH and enhancing nicotine absorption through the oral cavity. Advantageously, the compositions of present invention comprise nicotine in the intragranular portion which results in better stability to the product. Further the compositions of present invention comprise alkaline agent(s) in the extragranular portion while still achieving comparable dissolution and bioavailability to Commit®/Nicorette®) lozenges.

### SUMMARY OF THE INVENTION

An aspect of the present invention provides pharmaceutical compositions comprising nicotine or its derivatives as an active agent(s), and optionally one or more pharmaceutically acceptable excipient(s).

Another aspect of the present invention provides pharmaceutical compositions comprising nicotine polacrilex as an active agent(s), and optionally one or more pharmaceutically acceptable excipient(s).

An aspect of the present invention provides pharmaceutical compositions comprising nicotine polacrilex as an active agent(s), at least one alkaline agent, and optionally one or more other pharmaceutically acceptable excipient(s).

Another aspect of the present invention provides pharmaceutical compositions comprising nicotine polacrilex as an active agent(s), at least one alkaline agent in an amount of from about 0.1% w/w to about 99.0% w/w of the composition, and optionally one or more other pharmaceutically acceptable excipient(s).

An aspect of the present invention provides pharmaceutical compositions comprising nicotine polacrilex in the intragranular portion in an amount of from about 0.1% w/w to about 99.0% w/w of the composition, at least one alkaline agent in an amount of from about 0.1% w/w to about 99.0% w/w of the composition, and optionally one or more other pharmaceutically acceptable excipient(s).

Another aspect of the present invention provides pharmaceutical compositions comprising nicotine polacrilex in the intragranular portion in an amount of from about 0.1% w/w to about 99.0% w/w of the composition, at least one alkaline agent in the intragranular portion in an amount of from about 0.1% w/w to about 99.0% w/w of the composition, and optionally one or more other pharmaceutically acceptable excipient(s).

Yet another aspect of the present invention provides pharmaceutical compositions comprising nicotine polacrilex in the intragranular portion in an amount of from about 0.1% w/w to about 99.0% w/w of the composition, at least one alkaline agent in the extragranular portion in an amount of from about 0.1% w/w to about 99.0% w/w of the composition, and optionally one or more other pharmaceutically acceptable excipient(s).

An aspect of the present invention provides pharmaceutical compositions comprising nicotine polacrilex in the extragranular portion in an amount of from about 0.1% w/w to about 99.0% w/w of the composition, at least one alkaline agent in the intragranular portion in an amount of from about 0.1% w/w to about 99.0% w/w of the composition, and optionally one or more other pharmaceutically acceptable excipient(s).

Yet another aspect of the present invention provides pharmaceutical compositions comprising nicotine polacrilex in the extragranular portion in an amount of from about 0.1% w/w to about 99.0% w/w of the composition, at least one alkaline agent in the extragranular portion in an amount of from about 0.1% w/w to about 99.0% w/w of the composition, and optionally one or more other pharmaceutically acceptable excipient(s).

In an aspect, the present invention provides process for the preparation of pharmaceutical compositions, wherein the process comprises the following steps:
(1) treating nicotine or its derivatives with one or more intragranular excipients(s),

(ii) adding at least one alkaline agent(s) in extragranular portion optionally with one or more other pharmaceutically acceptable excipient(s), and

(iii) formulating the material of step (i) and (ii) into a suitable dosage form.

In an aspect, the present invention provides process for the preparation of pharmaceutical compositions, wherein the process comprises the following steps:
(1) treating at least one alkaline agent(s) with one or more intragranular excipients(s),

(ii) adding nicotine or derivatives thereof in the extragranular portion optionally with one or more other pharmaceutically acceptable excipient(s), and

(iii) formulating the material of step (i) and (ii) into a suitable dosage form.

An aspect of the present invention relates to method of using such compositions for reducing withdrawal symptoms or tobacco usage particularly nicotine craving, associated with quitting smoking.

### DETAILED DESCRIPTION OF THE INVENTION

The term “particle size” unless indicated otherwise in the specification relates to particles of nicotine or its derivative. Nicotine with specific “particle size” and distribution, or surface area would provide a fast dissolution of the active ingredient, would be easy to prepare and stable while maintaining the beneficial properties with respect to fast solubility and bioavailability. Particularly, according to the present invention, nicotine having an average particle size less than about 500 microns, and/or surface area less that about 5 m²/gm is useful.

The term “composition” or “pharmaceutical composition” or “dosage form” as used herein synonymously include solid dosage forms such as granules, multiunit particulate systems (MUPS), pellets, spheres, lozenges, mini-lozenges, chewing gums, tablets, capsules, mini-tablets, layered tablets, beads, particles, hard boiled candies, lollipops, or other device for transmucosal delivery and the like; and liquid dosage forms such as solutions, suspensions, emulsions, colloids and the like, meant for oral administration. In an embodiment the pharmaceutical composition is lozenges or mini-lozenges.

The term “excipient” means a pharmacologically inactive component such as a diluent, disintegrant, carrier, or the like. The excipients that are useful in preparing a pharmaceutical composition are generally safe, non-toxic and are acceptable for veterinary as well as human pharmaceutical use. Reference to an excipient includes both one and more than one such excipient.

The composition of the present invention includes nicotine. As used herein, the term “nicotine” refers to one or more compounds selected from: nicotine; derivatives of nicotine, such as nicotine salts and nicotine complexes; tobacco extract or leaf; any compounds or compositions that produce a similar physiological effect as nicotine, such as lobeline; and mixtures thereof. A variety of nicotine actives are well known in the art and are commercially available. Suitable nicotine actives for use herein include, but are not limited to, nicotine monotartrate, nicotine bitartrate, nicotine hydrochloride, nicotine dihydrochloride, nicotine sulfate, nicotine zinc chloride monohydrate, nicotine salicylate, nicotine oil, nicotine complexed with cyclodextrin, polymer resins such as nicotine polacrilex, and mixtures thereof. The nicotine active may be used in one or more distinct physical forms well known in the art, including free base forms, encapsulated forms, ionized forms and spray-dried forms.

Nicotine polacrilex is a resin complex of nicotine and polacrilin. Polacrilin or polacrilex resin is a weakly acidic cation-exchange resin prepared from methacrylic acid and divinylbenzene. Polacrilin resin is available as trade name Amberlite® IPR-64 supplied by the Dow chemical Company.

In an embodiment the nicotine is a nicotine resin complex. In another embodiment nicotine is nicotine polacrilex. In an embodiment the nicotine polacrilex is present in an amount from about 1 mg to about 10 mg of nicotine base per composition. In another embodiment the percentage of nicotine polacrilex per composition is from about 0.5 to about 20% of the total weight of the composition.

The present invention provides pharmaceutical compositions comprising nicotine or its derivatives as an active agent(s), and optionally one or more pharmaceutically acceptable excipient(s).

In an embodiment, present invention provides pharmaceutical compositions comprising nicotine polacrilex as an active agent(s), and optionally one or more pharmaceutically acceptable excipient(s).

In an embodiment, the present invention provides pharmaceutical compositions comprising nicotine polacrilex as an active agent(s), at least one alkaline agent, and optionally one or more other pharmaceutically acceptable excipient(s).

In another embodiment, the present invention provides pharmaceutical compositions comprising nicotine polacrilex as an active agent(s), at least one alkaline agent in an amount of from about 0.1% w/w to about 99.0% w/w of the composition, and optionally one or more other pharmaceutically acceptable excipient(s).

In an embodiment, the present invention provides pharmaceutical compositions comprising nicotine polacrilex in the intragranular portion in an amount of from about 0.1% w/w to about 99.0% w/w of the composition, at least one alkaline agent in an amount of from about 0.1% w/w to about 99.0% w/w of the composition, and optionally one or more other pharmaceutically acceptable excipient(s).

In another embodiment, the present invention provides pharmaceutical compositions comprising nicotine polacrilex in the intragranular portion in an amount of from about 0.1% w/w to about 99.0% w/w of the composition, at least one alkaline agent in the intragranular portion in an amount of from about 0.1% w/w to about 99.0% w/w of the composition, and optionally one or more other pharmaceutically acceptable excipient(s).

In yet another embodiment, the present invention provides pharmaceutical compositions comprising nicotine polacrilex in the intragranular portion in an amount of from about 0.1% w/w to about 99.0% w/w of the composition, at least one alkaline agent in the extragranular portion in an amount of from about 0.1% w/w to about 99.0% w/w of the composition, and optionally one or more other pharmaceutically acceptable excipient(s).

In an embodiment, present invention provides pharmaceutical compositions comprising nicotine polacrilex in the extragranular portion in an amount of from about 0.1% w/w to about 99.0% w/w of the composition, at least one alkaline agent in the intragranular portion in an amount of from about 0.1% w/w to about 99.0% w/w of the composition, and optionally one or more other pharmaceutically acceptable excipient(s).

In yet another embodiment, the present invention provides pharmaceutical compositions comprising nicotine polacrilex in the extragranular portion in an amount of from about 0.1% w/w to about 99.0% w/w of the composition, at least one alkaline agent in the extragranular portion in an amount of from about 0.1% w/w to about 99.0% w/w of the composition, and optionally one or more other pharmaceutically acceptable excipient(s).

In another embodiment, the present invention provides pharmaceutical compositions comprising nicotine polacrilex in the intragranular portion in an amount of from about 0.1% w/w to about 99.0% w/w of the composition, at least one alkaline agent in the intragranular portion in an amount of from about 0.1% w/w to about 99.0% w/w of the composition selected from but not limited to group comprising alkali metal carbonates or bicarbonates, alkali earth metal carbonates or bicarbonates used either alone or in combinations thereof, and optionally one or more other pharmaceutically acceptable excipient(s).

In another embodiment, the present invention provides pharmaceutical compositions comprising nicotine polacrilex in the intragranular portion in an amount of from about 0.1% w/w to about 99.0% w/w of the composition, at least one alkaline agent in the extragranular portion in an amount of from about 0.1% w/w to about 99.0% w/w of the composition selected from but not limited to group comprising alkali metal carbonates or bicarbonates, alkali earth metal carbonates or bicarbonates used either alone or in combinations thereof, and optionally one or more other pharmaceutically acceptable excipient(s).

In an embodiment, the present invention provides process for the preparation of pharmaceutical compositions, wherein the process comprises the following steps:
(1) treating nicotine or its derivatives with one or more intragranular excipients(s),

(ii) adding at least one alkaline agent(s) in extragranular portion optionally with one or more other pharmaceutically acceptable excipient(s), and

(iii) formulating the material of step (i) and (ii) into a suitable dosage form.

In an embodiment, the present invention provides process for the preparation of pharmaceutical compositions, wherein the process comprises the following steps:
(1) treating nicotine or its derivatives with one or more intragranular excipients(s) other than alkaline agent(s),

(ii) adding at least one alkaline agent(s) in extragranular portion, optionally with one or more other pharmaceutically acceptable excipient(s), and

(iii) formulating the material of step (i) and (ii) into a suitable dosage form.

In an embodiment, the present invention provides a pharmaceutical composition comprising: a) a master granule component comprising: nicotine or its derivatives and at least one or more intragranular excipients(s) other than alkaline agent(s), wherein the master granule component is obtained through wet or dry granulation; b) an extragranular component blended with the master granule component comprising at least one alkaline buffering agent selected from the group consisting of sodium carbonate, sodium bicarbonate, potassium phosphate, potassium carbonate and potassium bicarbonate, and mixtures thereof and c) compressing said mixture into oral dosage forms.

In an embodiment, the present invention provides a pharmaceutical composition comprising: a) a master granule component comprising: nicotine or its derivatives and at least one or more intragranular excipients(s) other than alkaline agent(s), wherein the master granule component is obtained through wet or dry granulation; b) an extragranular component blended with the master granule component comprising at least one alkaline buffering agent selected from the group consisting of sodium carbonate, sodium bicarbonate, potassium phosphate, potassium carbonate and potassium bicarbonate, and mixtures thereof and c) compressing said mixture into oral dosage forms selected from lozenges, mini-lozenges, chewing gums, tablets, bilayer tablets, inlaid tablets, tablet in tablet, multilayered tablets, mini-tablets filled in capsules and the like; beads, pellets presented in a sachet, capsule or tablet capsules such as soft and hard gelatin; sachets; granulates, microparticles, multiparticulates, powder.

In an embodiment, the present invention provides a pharmaceutical composition comprising: a) a master granule component comprising: nicotine or its derivatives and at least one or more intragranular excipients(s) other than alkaline agent(s), wherein the master granule component is obtained through wet or dry granulation; b) an extragranular component blended with the master granule component comprising at least one alkaline buffering agent selected from the group consisting of sodium carbonate, sodium bicarbonate, potassium phosphate, potassium carbonate and potassium bicarbonate, and mixtures thereof and c) compressing said mixture into oral dosage forms, wherein the nicotine containing oral lozenge dosage form is about 250 mg.

In an embodiment, the present invention provides process for the preparation of pharmaceutical compositions, wherein the process comprises of the following steps:
(1) treating at least one alkaline agent(s) with one or more intragranular excipients(s),

(ii) adding nicotine or derivatives thereof in the extragranular portion optionally with one or more other pharmaceutically acceptable excipient(s), and

(iii) formulating the material of step (i) and (ii) into a suitable dosage form.

In an embodiment, the present invention provides a nicotine containing oral lozenge dissolves in less than 15 minutes upon administration to the oral cavity.

In an embodiment, the present invention provides a nicotine containing oral dosage used for reducing withdrawal symptoms or tobacco usage particularly nicotine craving, associated with quitting smoking.

As used herein, unless indicated otherwise, references to total weight of the pharmaceutical composition refers to the total weight of the active agent(s) and pharmaceutically acceptable excipient(s).

In an embodiment the unit weight of lozenges is from about 100 mg to about 2000 mg. In another embodiment the unit weight of lozenges is from about 500 mg to about 1500 mg total. In yet another embodiment the unit weight of lozenges is about 300 mg to about 1300 mg.

“Pharmaceutically acceptable excipient(s)” are components that are added to the pharmaceutical formulation other than the active ingredient nicotine. Excipients may be added to facilitate manufacture, enhance stability, enhance product characteristics, enhance bioavailability, enhance patient acceptability, etc. Pharmaceutically acceptable excipient(s) includes but not limited to, one or more of diluents/fillers, binders, alkaline agent, buffers, salts, disintegrants, lubricants, glidants, compression aids, colors, sweeteners, preservatives, surfactants, suspending agents, dispersing agents, film formers, flavors, printing inks, and any other excipient known to the art for making pharmaceutical formulations. It will be appreciated by the person skilled in the art that a particular excipient may perform multiple roles in the pharmaceutical composition, such as for example, it can act as both a binder and filler, or as a binder and filler and disintegrant.

Binders hold the ingredients in the composition together. Exemplary binders are selected from but are not limited to, cellulose and its derivatives including ethyl cellulose, hydroxypropyl cellulose, hydroxypropyl methylcellulose, methylcellulose, hydroxyethyl cellulose, and carboxymethyl cellulose; gelatin, liquid glucose; starch and its derivatives (e.g. corn starch); sugars, polyvinyl pyrrolidone, agar, acacia, alginic acid or a salt thereof such as sodium alginate and potassium alginate, carbomer, carrageenan, chitosan, tragacanth, xanthan gum, guar gum, inulin, pectin, polycarbophil or salt thereof such as calcium polycarbophil, polyethylene glycol, polyethylene oxide, polyvinyl alcohol, pullulan and the like used either alone or combinations thereof. The binder may be used in the range of about 1 to about 15% by weight of the composition.

Diluents increase the bulk of the composition. Diluents according to the present invention are selected from but are not limited to lactose, sucrose, dextrose, mannose, fructose, galactose, maltose, trehalose, maltitol, sorbitol, mannitol, erythritol, xylitol, lactitol, isomalt, starlac, starch, modified starches, maltodextrin, dextrates, dextrin, dibasic calcium phosphate, tribasic calcium phosphate, calcium carbonate, calcium sulfate, powdered cellulose, microcrystalline cellulose, silicified microcrystalline cellulose, magnesium carbonate, magnesium oxide, magnesium alumino metasilicate and the like used either alone or in combinations thereof. The diluent may be used in the range of about 20% to about 98% by weight of the composition.

Alkaline agents according to the present invention include but are not limited to alkali metal carbonates or bicarbonates such as sodium carbonate, sodium bicarbonate, potassium carbonate and potassium bicarbonate and the like, alkali earth metal carbonates or bicarbonates such as magnesium carbonate, magnesium bicarbonate, calcium carbonate, calcium bicarbonate and the like, potassium phosphate, dibasic sodium phosphate, sodium borate, sodium aluminate, magnesium oxide, magnesium hydroxide, magnesium silicates and the like used either alone or combinations thereof. Alkaline agents may be used in the range of about 0.1% to about 10% by weight of the composition. In an embodiment, the ratio of alkali metal carbonates or bicarbonates to alkali earth metal carbonates or bicarbonates is 1:100 to 100:1.

Taste masking agents according to the present invention are selected from but are not limited to, sweetening agents and/or flavorants. Suitable intensive sweetening agents include but are not limited to, aspartame, acesulfame K, cyclamate and salts thereof, glycyrrhizin and salts thereof, neohesperidine, sucralose, saccharin and salts thereof, thaumatin and mixtures thereof. Suitable flavorants include but are not limited to, cherry, cinnamon, menthol, peppermint, wintergreen, mint, sweet mint, spearmint, vanillin, chocolate, coffee, cinnamon, clove, tobacco, citrus and fruit flavors and the like used either alone or combinations thereof.

Disintegrants according to the present invention are selected from but are not limited to, cellulose and its derivatives including low-substituted hydroxypropyl cellulose, cross-linked polyvinylpyrrolidone, sodium carboxymethylcellulose, cross-linked sodium carboxymethylcellulose, microcrystalline cellulose, sodium starch glycolate, ion-exchange resins, starch and modified starches including □lavor□ne□ised starch, formalin-casein, used either alone or in combinations comprising one or more of the foregoing disintegrants. In an embodiment, the disintegrant may be used in the range of about 1 to about 20% by weight of the composition.

Lubricants and glidants aid in the processing of powder materials. Exemplary lubricants are selected from but are not limited to, calcium stearate, magnesium stearate, zinc stearate, stearic acid, glycerol behenate, mineral oil, polyethylene glycol, sodium stearyl fumarate, talc, and vegetable oil, and the like used either alone or in combinations thereof. Exemplary glidants include but are not limited to, talc, silicon dioxide, colloidal silicon dioxide, cornstarch and the like used either alone or in combination thereof.

“Suitable solvent” according to the present invention can be any solvent in which the binder is soluble or dispersible and is selected from isopropyl alcohol, ethanol, water, acetone, methylene chloride and the like or mixtures thereof.

It must be appreciated that the pharmaceutical compositions of the present invention can include all the dosage forms known to a person skilled in art, viz. formulations such as single unit dosage forms in the form of lozenges, mini-lozenges, chewing gums, tablets, bilayer tablets, inlaid tablets, tablet in tablet, multilayered tablets, mini-tablets filled in capsules and the like; beads, pellets presented in a sachet, capsule or tablet capsules such as soft and hard gelatin; sachets; granulates, microparticles, multiparticulates, powder and the like. The tablets or lozenges in accordance with the present invention can be prepared by either direct compression, dry compression (slugging), or by wet granulation. The wet granulation method may comprise use of aqueous solvent such as water or an organic solvent such as ethanol, or a mixture thereof as the granulating aid.

Additional excipients such as stabilizing aids may also be included in the composition are selected from but are not limited to, chelating agents, anti-oxidants or preservatives. Antioxidants/preservatives suitable for use in the present invention may include sodium benzoate, butyl-hydroxy toluene and tocopherol and its salts.

In an embodiment, the compositions of the present invention may additionally comprise of a colorant in order to produce a desirable color. Colors known to be ‘FD&C’ certified may be used to provide coloring to the product and are within the purview of the present invention.

Suitable colorants include natural colorants i.e., pigments and dyes obtained from mineral, plant, and animal sources. Examples of natural colorants include red ferric oxide, yellow ferric oxide, annattenes, alizarin, indigo, rutin, quercetin, and the like. Synthetic colorants may also be used, which is typically an FD&C or D&C dye, e.g., an approved dye selected from the so-called ‘coal-tar’ dyes, such as a nitroso dye, a nitro dye, an azo dye, an oxazine, a thiazine, a pyrazolone, a xanthene, an indigoid, an anthraquinone, an acridine, a rosaniline, a phthalein, a □lavor□ne, or a ‘lake’ thereof, i.e. an aluminum or calcium salt thereof. Particularly preferred colorants are food colorants in the ‘GRAS’ (Generally Regarded as Safe) category.

In an embodiment, the tablet compositions of the present invention may be film coated. A film forming agent may provide smooth film-forming coating suspensions and enhance the rheological mechanical strength properties of film coating gel matrices. Film forming agents include, for example, polyvinylpyrrolidone, natural gums, starches, and cellulosic polymers. A cellulosic polymer may include a molecule comprising at least one cellulose polymer or derivative modified with small amounts of propylene glycol ether groups attached to the cellulose anhydroglucose chain affording binding properties that enhance the reinforcing film properties of film applications. Examples of cellulosic polymers include, but are not limited to, hydroxypropyl methyl cellulose (“HPMC”), carboxymethyl cellulose (“CMC”) or salts thereof, hydroxypropyl cellulose (“HPC”), methylcellulose (“MC”), hydroxyethyl cellulose (“HEC”), and the like. In addition, cellulosic polymers may be characterized as ionic or non-ionic. Ionic cellulosic polymers include, for example, sodium CMC. Non-ionic cellulosic polymers include, for example, HPMC, HPC, HEC, and MC. Varieties of commercially available cellulosic polymers exist and may include, for example, Spectracel® HPMC compositions (available from Sensient Technologies). Further, other commercially available coating materials are available marketed under the brand name Opadry® for example Opadry II Gray which contains: lactose monohydrate NF, hypromellose type 2910 USP, titanium dioxide USP, triacetin USP, and iron oxide black JPE; Opadry II Pink which contains: hypromellose type 2910 USP, titanium dioxide USP, lactose monohydrate NF, polyethylene glycol 3350 NF, triacetin USP, and FD&C Red #40; Opadry II Blue which contains: hypromellose type 2910 USP, lactose monohydrate NF, FD&C Blue #1, polyethylene glycol 3350 NF, FD&C Blue #2, titanium dioxide USP, triacetin USP, and D&C Yellow #10; Opadry II Yellow which contains: hypromellose type 2910 USP, lactose monohydrate NF, titanium dioxide USP, iron oxide yellow NF, polyethylene glycol 3350 NF, and triacetin USP; Opadry II Purple which contains: hypromellose type 2910 USP, lactose monohydrate NF, titanium dioxide USP, D&C Red #27, polyethylene glycol 3350 NF, triacetin USP, and FD&C Blue #1 and the like.

In another embodiment, the present invention provides a method of using such compositions for reducing withdrawal symptoms or tobacco usage particularly nicotine craving, associated with quitting smoking.

The compositions of the present invention can be packed into suitable containers such as bottles, blisters or pouch. Further, the packages may optionally contain a desiccant or an antioxidant or oxygen absorbent or combinations thereof.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

### Examples—1 to 2

| | | | |
|---|---|---|---|
| | | Example 1 | Example 2 |
| S. No. | Ingredients | % age (w/w) | % age (w/w) |

Intragranular Materials

| | | | |
|---|---|---|---|
| 1. | Mannitol | 79.04 | 79.04 |
| 2. | Calcium polycarbophil | 2.65 | 2.65 |
| 3. | Xanthan gum | 1.03 | 1.03 |
| 4. | Sodium alginate | 5.31 | 5.31 |
| 5. | Potassium bicarbonate | 0.23 | — |
| 6. | Sodium carbonate | 4.31 | — |

Binder Solution

| | | | |
|---|---|---|---|
| 7. | Purified water* | q.s. | q.s. |

Extragranular Materials

| | | | |
|---|---|---|---|
| 8. | Nicotine polacrilex | 1.67 | 1.67 |
| 9. | Mannitol | 3.80 | 3.80 |
| 10. | Potassium bicarbonate | — | 0.23 |
| 11. | Sodium carbonate | — | 4.31 |
| 12. | Colloidal silicone dioxide | 0.30 | 0.30 |
| 13. | Aspartame | 0.57 | 0.57 |
| 14. | Flavor | 0.10 | 0.10 |
| 15. | Magnesium stearate | 1.00 | 1.00 |

*Lost in processing
q.s.—quantity sufficient

Brief Manufacturing Procedure:

Granulation:
Step 1: Sift the intragranular materials through ASTM #30 mesh.
Step 2: Granulate the materials of step 1 using purified water as a binder solution.
Step 3: Wet mill the granules of step 2 using suitable screen size.
Step 4: Dry the wet granules of step 3.
Step 5: Mill the dried granules of step 4 using suitable screen size.

Blending:
Step 6: Co-sift the extragranular materials through ASTM #40 mesh.
Step 7: Blend the half quantity of dried & milled granules of step 5 and sifted materials of step 6 in the blender.
Step 8: Add remaining half quantity of granules of step 5 to the step 7 and mix.
Step 9: Sift the magnesium stearate through ASTM #60 mesh.
Step 10: Add the sifted magnesium stearate of step 9 into the step 8 and lubricate.

Compression:
Step 11: Compress the lubricated granules of step 10 using suitable tooling.

Packing:
Step 12: Pack the tablets of step 11 in suitable pack.

### Examples—3 to 5

| | | | | |
|---|---|---|---|---|
| | | Example 3 | | Example 5 |
| S. | | % age | Example 4 | % age |
| No. | Ingredients | (w/w) | % age (w/w) | (w/w) |

Intragranular Materials

| | | | | |
|---|---|---|---|---|
| 1. | Mannitol | 81.73 | 79.92 | 81.73 |
| 2. | Calcium polycarbophil | 2.65 | 2.65 | 2.65 |
| 3. | Xanthan gum | 1.50 | 2.00 | 1.50 |
| 4. | Sodium Alginate | 2.65 | 2.65 | 2.65 |
| 5. | Potassium bicarbonate | 0.23 | — | 0.23 |
| 6. | Sodium carbonate | 1.90 | — | 1.90 |
| 7. | Dibasic sodium phosphate | — | 2.00 | — |
| 8. | Sodium borate | 2.00 | — | — |
| 9. | Sodium aluminate | — | — | 2.00 |

Binder Solution

| | | | | |
|---|---|---|---|---|
| 10. | Purified water* | q.s. | q.s. | q.s. |

Extragranular Materials

| | | | | |
|---|---|---|---|---|
| 11. | Nicotine polacrilex | 1.67 | 1.67 | 1.67 |
| 12. | Mannitol | 3.80 | 5.48 | 3.80 |
| 13. | Potassium bicarbonate | 0.23 | — | 0.23 |
| 14. | Sodium carbonate | — | 1.67 | — |
| 15. | Colloidal silicone dioxide | 0.30 | 0.30 | 0.30 |
| 16. | Aspartame | 0.57 | 0.57 | 0.57 |
| 17. | Flavor | 0.10 | 0.10 | 0.10 |
| 18. | Magnesium stearate | 1.00 | 1.00 | 1.00 |

*Lost in processing
q.s.—quantity sufficient

Brief Manufacturing Procedure:

Granulation:
Step 1: Sift the intragranular materials through ASTM #30 mesh.
Step 2: Granulate the materials of step 1 using purified water as a binder solution.
Step 3: Wet mill the granules of step 2 using suitable screen size.
Step 4: Dry the wet granules of step 3.
Step 5: Mill the dried granules of step 4 using suitable screen size.

Blending:
Step 6: Co-sift the extragranular materials through ASTM #40 mesh.
Step 7: Blend the half quantity of dried & milled granules of step 5 and sifted materials of step 6 in the blender.
Step 8: Add remaining half quantity of granules of step 5 to the step 7 and mix.
Step 9: Sift the magnesium stearate through ASTM #60 mesh.
Step 10: Add the sifted magnesium stearate of step 9 into the step 8 and lubricate.

Compression:
Step 11: Compress the lubricated granules of step 10 using suitable tooling.

Packing:
Step 12: Pack the tablets of step 11 in suitable pack.

### Examples—6 to 9

| | | | | | |
|---|---|---|---|---|---|
| | | Example 6 | Example 7 | Example 8 | Example 9 |
| S. | | % age | % age | % age | % age |
| No. | Ingredients | (w/w) | (w/w) | (w/w) | (w/w) |

Intragranular Materials

| | | | | | |
|---|---|---|---|---|---|
| 1. | Nicotine | 1.67 | 1.67 | 1.67 | 1.67 |
| | polacrilex | | | | |
| 2. | Mannitol | 81.83 | 83.13 | 82.9 | 79.8 |
| 3. | Calcium | 2.33 | 2.65 | 2.65 | 2.65 |
| | polycarbophil | | | | |
| 4. | Xanthan gum | 1.03 | 1.03 | 1.03 | 1.03 |
| 5. | Colloidal silicone | 0.26 | 0.20 | 0.2 | 0.2 |
| | dioxide | | | | |
| 6. | Sodium alginate | 0.83 | 5.31 | 5.31 | 5.31 |

Binder Solution

| | | | | | |
|---|---|---|---|---|---|
| 7. | Purified water | q.s. | q.s. | q.s. | q.s. |

Extra-granular Materials

| | | | | | |
|---|---|---|---|---|---|
| 8. | Mannitol | 2.86 | 2.03 | 2.03 | 2.03 |
| 9. | Sodium alginate | 5.31 | — | — | — |
| 10. | Potassium | 0.23 | 0.23 | — | 5.35 |
| | bicarbonate | | | | |
| 11. | Sodium carbonate | 1.79 | 1.79 | 2.25 | — |
| 12. | Colloidal silicone | 0.20 | 0.30 | 0.30 | 0.30 |
| | dioxide | | | | |
| 13. | Aspartame | 0.57 | 0.57 | 0.57 | 0.57 |
| 14. | Flavor | 0.10 | 0.10 | 0.10 | 0.10 |
| 15. | Magnesium | 1.00 | 1.00 | 1.00 | 1.00 |
| | stearate | | | | |

*Lost in processing
q.s.—quantity sufficient

Brief Manufacturing Procedure:

Granulation:
Step 1: Sift the intragranular materials through ASTM #40 mesh.
Step 2: Granulate the materials of step 1 using purified water as a binder solution.
Step 3: Wet mill the granules of step 2 using suitable screen size.
Step 4: Dry the wet granules of step 3.
Step 5: Mill the dried granules of step 4 using suitable screen size.

Blending:
Step 6: Co-sift mannitol, sodium alginate, colloidal silicon dioxide through ASTM #40 mesh.
Step 7: Sift sodium carbonate and/or potassium bicarbonate through ASTM #60 mesh.
Step 8: Blend the half quantity of dried granules of step 5 and sifted materials of step 6 & 7 using suitable blender.
Step 9: Sift the aspartame and flavor through ASTM #60 mesh.
Step 10: Blend the sifted materials of step 9, remaining half quantity of dried granules of step 5 and the mixture of step 8 into the blender.
Step 11: Sift the magnesium stearate through ASTM #60 mesh
Step 12: Add the sifted magnesium stearate of step 11 to step 10 and lubricate.

Compression:
Step 13: Compress the lubricated granules of step 12 using suitable tooling.

Packing:
Step 14: Pack the tablets of step 13 in suitable pack.

### Example—10

| | | |
|---|---|---|
| | | Example 10 |
| S. No. | Ingredients | % age (w/w) |

Intragranular Materials

| | | |
|---|---|---|
| 1. | Nicotine polacrilex | 1.67 |
| 2. | Mannitol | 76.58 |
| 3. | Sodium alginate | 8.19 |
| 4. | Colloidal silicone dioxide | 0.26 |

Binder Solution

| | | |
|---|---|---|
| 5. | Purified water | q.s. |

Extra-granular Materials

| | | |
|---|---|---|
| 6. | Mannitol | 2.86 |
| 7. | Sodium alginate | 7.55 |
| 8. | Potassium bicarbonate | 0.23 |
| 9. | Sodium carbonate | 1.79 |
| 10. | Colloidal silicone dioxide | 0.2 |
| 11. | Aspartame | 0.57 |
| 12. | Flavor | 0.1 |
| 13. | Magnesium stearate | 1 |

*Lost in processing
q.s.—quantity sufficient

Brief Manufacturing Procedure:

Manufacturing Process is similar to Example 6.

### Example—11

| | | |
|---|---|---|
| | | Example 11 |
| S. No. | Ingredients | % age (w/w) |

Intragranular Materials

| | | |
|---|---|---|
| 1. | Nicotine polacrilex | 1.67 |
| 2. | Mannitol | 75.45 |
| 3. | Sodium alginate | 8.19 |
| 4. | Colloidal silicone dioxide | 0.26 |

Binder Solution

| | | |
|---|---|---|
| 5. | Purified water | q.s. |

Extra-granular Materials

| | | |
|---|---|---|
| 6. | Mannitol | 2.86 |
| 7. | Sodium alginate | 7.55 |
| 8. | Magnesium carbonate | 0.5 |
| 9. | Sodium carbonate | 1.65 |
| 10. | Colloidal silicone dioxide | 0.2 |
| 11. | Aspartame | 0.57 |
| 12. | Flavor | 0.1 |
| 13. | Magnesium stearate | 1 |

*Lost in processing
q.s.—quantity sufficient

Brief Manufacturing Procedure:

Granulation:
Step 1: Sift the intragranular materials through ASTM #40 mesh.
Step 2: Granulate the materials of step 1 using purified water as a binder solution.
Step 3: Wet mill the granules of step 2 using suitable screen size.
Step 4: Dry the wet granules of step 3.
Step 5: Mill the dried granules of step 4 using suitable screen size.

Blending:
Step 6: Co-sift mannitol, sodium alginate, colloidal silicon dioxide through ASTM #40 mesh.
Step 7: Sift sodium carbonate and magnesium carbonate through ASTM #60 mesh.
Step 8: Blend the half quantity of dried granules of step 5 and sifted materials of step 6 & 7 using suitable blender.
Step 9: Sift the aspartame and flavor through ASTM #60 mesh.
Step 10: Blend the sifted materials of step 9, remaining half quantity of dried granules of step 5 and the mixture of step 8 into the blender.
Step 11: Sift the magnesium stearate through ASTM #60 mesh
Step 12: Add the sifted magnesium stearate of step 11 to step 10 and lubricate.

Compression:
Step 13: Compress the lubricated granules of step 12 using suitable tooling.

Packing:
Step 14: Pack the tablets of step 13 in suitable pack.

### Examples—12 to 15

| | | Example | Example | Example | Example |
|---|---|---|---|---|---|
| | | 12 | 13 | 14 | 15 |
| S. | | % age | % age | % age | % age |
| No. | Ingredients | (w/w) | (w/w) | (w/w) | (w/w) |
| 1. | Nicotine | 1.67 | 1.67 | 1.67 | 1.67 |
| | polacrilex | | | | |
| 2. | Mannitol | 21.00 | 18.00 | 21.00 | 21.00 |
| 3. | Sorbitol | 70.5 | — | 70.1 | 67.25 |
| 4. | Xylitol | — | 73.5 | — | — |
| 5. | Povidone | 3.00 | 3.00 | 3.00 | 3.00 |
| 6. | Potassium | 0.23 | 0.23 | — | 5.25 |
| | bicarbonate | | | | |
| 7. | Sodium carbonate | 1.79 | 1.79 | 2.35 | — |
| 8. | Colloidal silicone | 0.20 | 0.30 | 0.20 | 0.20 |
| | dioxide | | | | |
| 9. | Aspartame | 0.57 | 0.57 | 0.57 | 0.57 |
| 10. | Flavour | 0.10 | 0.10 | 0.10 | 0.10 |
| 11. | Magnesium | 1.00 | 1.00 | 1.00 | 1.00 |
| | stearate | | | | |

Brief Manufacturing Procedure:
Step 1: Co-sift nicotine polacrilex and mannitol through ASTM #40 mesh.
Step 2: Sift sorbitol/xylitol, povidone, colloidal silicon dioxide through ASTM #40 mesh.
Step 3: Blend materials of step 2 and step 1 using suitable blender.
Step 4: Sift sodium carbonate and/or potassium bicarbonate, aspartame and □lavor through ASTM #60 mesh.
Step 5: Add materials of step 4 to step 3 into the blender and mix.
Step 6: Sift the magnesium stearate through ASTM #60 mesh
Step 7: Add the sifted magnesium stearate of step 6 to step 5 and lubricate.

Compression:
Step 8: Compress the lubricated mixture of step 7 using suitable tooling.

Packing:
Step 9: Pack the tablets of step 8 in suitable pack.

We claim:

1. A pharmaceutical composition comprising alkaloid or its derivatives, at least one alkaline agent and optionally one or more pharmaceutically acceptable excipient(s).

2. The composition of claim 1, wherein alkaloid is a potent parasympathomimetic alkaloid.

3. The composition of claim 1, wherein alkaloid is Nicotine or Nicotine polacrilex in an amount of from about 0.1% w/w to about 99.0% w/w of the composition.

4. The composition of claim 1, wherein alkaline agent in an amount of from about 0.1% w/w to about 99.0% w/w of the composition.

5. A pharmaceutical composition comprising: a) a master granule component comprising: nicotine or its derivatives and at least one or more intragranular excipients(s) other than alkaline agent(s), wherein the master granule component is obtained through either wet or dry granulation; b) an extragranular component blended with the master granule component comprising at least one alkaline buffering agent selected from the group consisting of sodium carbonate, sodium bicarbonate, potassium phosphate, potassium carbonate and potassium bicarbonate, and mixtures thereof and c) compressing said mixture into oral dosage forms.

6. The nicotine containing composition with improved user compliance prepared by the process of claim 5, wherein unit weight of lozenges is from about 100 mg to about 2000 mg.

7. The nicotine containing composition with improved user compliance prepared by the process of claim 5, wherein the unit weight of the oral lozenge dosage form is about 250 mg.

8. The nicotine containing composition with improved user compliance prepared by the process of claim 5, wherein the oral lozenge dissolves in less than 15 minutes upon administration to the oral cavity.

9. The nicotine containing composition of claim 5, further comprising diluents/fillers.

10. The nicotine containing composition of claim 9, wherein diluents/fillers selected from group consisting of lactose, sucrose, dextrose, mannose, fructose, galactose, maltose, trehalose, maltitol, sorbitol, mannitol, erythritol, xylitol, lactitol, isomalt, starlac, starch, modified starches, maltodextrin, dextrates, dextrin, dibasic calcium phosphate, tribasic calcium phosphate, calcium carbonate, calcium sulfate, powdered cellulose, microcrystalline cellulose, silicified microcrystalline cellulose, magnesium carbonate, magnesium oxide, magnesium alumino metasilicate and the like used either alone or in combinations thereof.

11. The nicotine containing composition of claim 5, further comprising a binder.

12. The nicotine containing composition of claim 11, wherein binder selected from group consisting of cellulose and its derivatives including ethyl cellulose, hydroxypropyl cellulose, hydroxypropyl methylcellulose, methylcellulose, hydroxyethyl cellulose, and carboxymethyl cellulose; gelatin, liquid glucose; starch and its derivatives (e.g. corn starch); sugars, polyvinyl pyrrolidone, agar, acacia, alginic acid or a salt thereof such as sodium alginate and potassium alginate, carbomer, carrageenan, chitosan, tragacanth, xanthan gum, guar gum, inulin, pectin, polycarbophil or salt thereof such as calcium polycarbophil, polyethylene glycol, polyethylene oxide, polyvinyl alcohol, pullulan and the like used either alone or combinations thereof.

13. The nicotine containing composition of claim 5, further comprising disintegrants.

14. The nicotine containing composition of claim 13, wherein disintegrant is selected from group consisting cellulose and its derivatives including low-substituted hydroxypropyl cellulose, cross-linked polyvinylpyrrolidone, sodium carboxymethylcellulose, cross-linked sodium carboxymethyl cellulose, microcrystalline cellulose, sodium starch glycolate, ion-exchange resins, starch and modified starches including pregelatinized starch, formalin-casein, used either alone or in combinations thereof.

15. The nicotine containing composition of claim 5, further comprising lubricant.

16. The nicotine containing composition of claim 15, wherein lubricant selected from group consisting calcium stearate, magnesium stearate, zinc stearate, stearic acid, glycerol behenate, mineral oil, polyethylene glycol, sodium stearyl fumarate, talc, and vegetable oil, and the like used either alone or in combinations thereof. Exemplary glidants include but are not limited to, talc, silicon dioxide, colloidal silicon dioxide, cornstarch and the like used either alone or in combination thereof.

17. The nicotine containing composition of claim 5, wherein flavorants are selected from group consisting cherry, cinnamon, menthol, peppermint, wintergreen, mint, sweet mint, spearmint, vanillin, chocolate, coffee, cinnamon, clove, tobacco, citrus and fruit flavors and the like used either alone or combinations thereof.

18. The nicotine containing composition of claim 5, wherein other pharmaceutically acceptable excipients are buffers, salts, glidants, compression aids, colors, sweeteners, preservatives, surfactants, suspending agents, dispersing agents, film formers, printing inks.

19. The dosage form of claim 5, selected from lozenges, mini-lozenges, chewing gums, tablets, bilayer tablets, inlaid tablets, tablet in tablet, multilayered tablets, mini-tablets filled in capsules and the like; beads, pellets presented in a sachet, capsule or tablet capsules such as soft and hard gelatin; sachets; granulates, microparticles, multiparticulates, powder.

20. The nicotine containing composition of claim 5, used for reducing withdrawal symptoms or tobacco usage particularly nicotine craving, associated with quitting smoking.
